SEQUENCE LISTING (24 sequences)

SEQ ID NO: 1 - DNA, 16101 bp, Canis familiaris
gctccatgcagggagcctgatgtggcacttgatcctgggactccaggatcatgccctgagctgaaggcaggtgcttacccgctgagccacccaggcatcccaataaaatcttttaaaaataaataaataaaatactcttggtgtaatgttatgaaactggactatttatacataatcgtatagtataagatttaggcagtgatttctaaattgcccatatagtaaaaattatatataaactatattatatataatatatgacatataaagaaatgttacatatagatatagataactagatatatgtcttcctgaatcttaatacaatgagtatttctaaatgtttacatttgaaaaaaatccacaggaaattatgaggaacacttaagattgagaaacattgcccaaaaggatattaatctgcattgatatttctattgtggaataacaaaaattgaaggatgggacacattagtgtatgttaaatcacttcgaaagatagatgcttattaaatatgtgtaatagatcatgccttgctacctgaaagttggaataatgcaaacttattcctttaaataaagactattcattacaagtaatttttttaaaacttgacaatggtttgatgttagaatgcttttaatttcaagtaaatccatccaacagtggctgaaacaaataagggattgcttttttgtgcctgtctcttcaaattacaaggcatctggagagagctagctactggcagtggttcaacagctcagctgcatcacggtgactgtcagatcttctcagcttttacttacatatatacaaggtgaattgcaaacttaggcatcatacccatggattttaaggcagaaggaaagagcagtagcttcggggtactctctaaagcaaacgctcttcaaaaagcctctgcttatgtttcatttgtctagtgctgtgttaaaaagctatccttggataaaagggaggctggggagatcctaaaaatccctaggcattgtaaatgaaatacctaaaggaaataaagtgctctacagactgcctaatagataataagactgccaaaagaggcgttataaaaccaaaaataacaacaatagcaacaaaaccccaaaaaacaaaaaactgtactcaggattttcagttgtatggtagcaatatctttcattgtatcaccatgaaaattaaataaaacaatttcagaaaaatttattgagctcttacagatatcaggtagccttggctttggagacaagttagaatagtacagagtctctattcacagagctcactttctaatagggaaactgagtgtaaatcaatagattcaagaattgataaatccaggaagaagacagaagtaggttaaattgaagaaagggatttatggttgccagggagagtggtgggtaacaatggttagggaagatctttctgaggcagaggaagaccagattattaggaagtaggacattcaaacaaaagttttatccaggaaagggggttggattttgtgtgtaaaaacaaatttaagtgagtgtggctaaaaatcaattcatagagtaaagagtagtcatctgagccaactcaggcaaaccctgcctacaattaggggcctatatttgggtatagtggaacaccattggttaatattttaatcagtggggtttatgatttaaggaatgctttttttttagagataatgtttattttattttactttttaagatttttatttatttatgagagacacagagggagagagagagagaggccgagacacaggcagagggagaagcaggctccattcagggagccccatgtgggactcaatcccgggtctccaggatcacgccttgggtcaaaggcaggcgccaaactgctgagccacccagggatctctaaggaatgctttttaaaaagataaccatagcttccctgtagaagatagaataaaagaatgcaatattagaggcagggagaccaatatagctgggaaatagaaatggctcagattgtagaagtggtaataattagattcaagatatattttgaaggtatatcctcctggatcttctatttattagatgtacactatgaaggaagtagatgaattcaagttgacttctagattttcagacaaaacaactgagtgaaaaagagtgtcatttactgagattagtgaaagcttgtaggaagagtcatttgggggcatagggaggaaagtagctctgctcttgccaatataattttgatgtgcgtatagtactcccaaaagcaagcacaaaatagagggtatgcatttatgactatgcagctcaggagagattttttagattaaaccattcaaaccactgtagcagggcacaaaagtgaaaaagaactgtgtgcttgaaatggtcctaaccatcaaccattaataaaagaaaaggagtccacaagtctagaaatgtgggcaaagattcacaactgcagacaggctcttcctgaaatctgtagcatagaactaacgatgaggacacaaaggtggtcaaccacagtgaaacccacaaaagtagaataaaaaatgtgtgaagtgtgtatgtaaacacacgaaaagcaaagttgaaagcatttttccagtgactgtgatagaagagtctccagtgaattgctcatgtaataaagtcaatggcattagaagaggaattgtctccgtgttggtccaggcctttcgtcagtttgaatagttagtgcacaacatttcatgtgcagttgtcatgtcgcaaagggacaccacgtgacttgtgggacatctctatcaagaaaccaacgaatcactccacagatgctgttcagagtgcttgcttgctctgatggctgccctacagtgtctacttaaaggcacctcagtggatccgccacacaaactgactagctactaaaagaatacttgtgaatgatgtgaaaaaaaatcccaaaacttctgttcaaaccaatttggttgatcctttcatattctgaagcacacaaagaaggtttcctgggagtaatttctgttcagtgtccacatttcataacatttattggatttatgcaattctcttgactcccccctgcccccaaagcactttcatatgtgggcttactttagactcaggacacttcacggagatggataggatagccatattcatctgcttttctgagccagaagcttcttcagaatgggaaatacttaaactatagccaaagaaacatccattaatgacctgtggaggaccggtttcagttggaactacctgcatctggtcatatcctaacaccctaaaagtatgttctttggcaaaattacttccaatattagggatttggaattgcagccaaacattcttacatatacagatatactgacctggcagagatgttacaggaagggggtgaaatgtaaatccatacggcagctatacaatttacgtattttgcaagatgatgtattcctccttaggaaattgagaaataatagttacttcaaattatcctcgaagatcatactttattaaaactaactgaacgattaaatttataatatttttctcctatacaataattaaaagctgtggctgcttctcaaaaaaaaaaggaggggggaagagggggaagagaagaagaaaataaatttcaaaaagattttgcctttgtttagatatttttgtggtacctttcattaatggccataaataaaaacaattcctgattatttttcttcaaagttttaatttttctctttatttaattataattagttgtgatcttcattttctttcatatgaataagagaaagaaattttgaaagaaatttttgaaatttggaaagaaagaaaacagcactacagagaaactgcatactgcatgtcttcttcttggaaatcaatatttaaacaaatcaacccatagacatatagcttgaaaattacagatttgtttttaagcttcctgtttttaagcctctaagactgaggtaccagaagcacagttagctattgaaaaatctttacctcagaaaaaaaaatttattccctgtcctcttttgccacacagtttgcgtgtgtttatgtgtgttttttaatgtagagcccagttgtgacatatttgttttcttttgtattctcccaactggctgaaaattagtcagatgaataatctggcgtcagtcttctggtcccttttctgaagcctggtattagcagtccagctcatttaaatctctccttcactctaacttcaagctctctctgcagaactgtacctgagcaccatgtgctacttagttgcctaaaagaaaaactcttttgcacagaaaaattacatttattaaggaacacacctaaatactatcatttctgataaaagtgacagttggaatcatttgagaaccatgcagaatttttctggcagcagttacccttaacaaatgtactgcgatgctgtgtgctggatgtagattcccatggaaaagggagtagctccaaaatcctaatagtaccatgtggtctggccatgaactaaagcttttcaaaccatatggaaactcattacttcaaagcacttccacagtcaccttattttattattattttcacattagcgctatagcataagggtaaagtggaaaaatattcccagcttatagatggggagacgaacatgcagcaatcattgttaatttccttatttctcctaatgtcaccaatattctcttggaagtttacctgttcattactttttaatagaatcgattgagctattactaggatttattctttcccctccatttctggcattaacattctagaccacaatttcttcccacatgcctagattacttccataactttctagatttctccctacctatgttctcttctccttttaggttgtgttcagaattgatatagttataaaagtttggctcagtaaaaaaaacacaagaatgttccaaagaaagggaacatcagcctggagtgttcctgaggaactacaaaatgtttagcttccctggagattgtcatagaaatgtaaagggttgtgtaagatgagaccataagataaagtgaggagcaggttatatatcggcttttacattccatttcaaggaatgcttatgctgaaagcaaggacattttaagagctttaggtctgaaaattgcataatcaaatgtggatgcttacaaaatcaaccacactcatgctgtaaaatgaagaaaataaattagtccattgggagactgtacagtttgggatgggaggtaattaatttggaagcctgtcagtaatcaggccaagtgattatggtattctgtactaaagtgctggcaatggggtgagtaaaggtaagttgctttaaaaatatagaggaaatagacttaaaacacatgagaagtaacctattaaaaaatcaaaaataattcataaactctaacttaagcactggtttttttttaatatttttatttatttatgatagtcacagagagagagagaggcagagacacaggcagagggagaagcaggctccatgccaggagcctgatgtgggattcgatcccgggtctccaggatcgtgccccgggccaaaggcaggcgccaaaccgctgcaccacccagggatcccaagcactggtttttaatggtgatttttcctcaatagcgaacaacaagagttaggttctaacttggaaatgatgagcctggaatgattgggtacatccagatggggatgtctccaggacagtttaatgtacatgactgagggtcaagaaagatatacacttaggtgtcatctgtataaatggagcagctgttgaatgattacacatatggttgtgtgttatatgcaggatgttaaaaagggtgctgtttattttatctatcttctctacttggggcaccaatttggggtcaaatatataaaacgattctttatccaaagtgggtaagtttaattttactaaagcgatgaaaaaggtggtataaagcctaaaaattaaatatacataggtacttatgatgaacaagacactgttcgaaatcacttgctttttgtgccatgtaatccacttagaaactcaacagtagtccccactttacctcaaagacataaaataaaggaccatcatttacctagaagtccaagaatggagccaaatcatgtatacaatttcttgaaacagcatgattaattcacatttattaatgattactctatgtctagcatgacactagatgctgtgtactgaagtctcttttttagtttatattaggttacactacataaccccaaataatgaatggcacagtgaaatgtaaacataaatacatatatgcacaggtgcccaaaggaatctacaactcattggcagctaagctcagtgtatttcagtccctcttttctgagaagcacatagaagggaaagccagccacctgctcccagtgcccctgaaccctgatccagaaacagcctgacctggtgggatatgaaaagtttattctctaatagtgaacaaggtttcaaaagagcttcggtgaactcctgtgatttcctatccacatttcattcttaaggtaattctatttgaatggccttttcaaaatttaagttctatattctcatgctatttcctcatctggtttgttcagttggacactcaaaggtcttgttctgattccccttacctagttacatttgataaattggcatttcgccctcacttcagctaggctgaattacactttgcattgctgatctttctggggttgggggttcatgcatttctttagtctccaggctgctcctttgcttctctactctgagatccattccccactctccctgctttgctctgtcatggaagtccgaatgccgcacactgcaattctggatggtgctaattagcaaatgggaagtgttagcagtacgaggaggaagggacaatctagggtattttttcccttcacttggatttgggcgccagcctctggggtggccgtgtctcccccaggattctagattttactggatgtgctgctgactcctgacccctgttctttctatctcctttaactctcctgcccaggggtgttactagccctctgctttgttaatctctggtttgtttcatttgtcgtctgattggcttttcagccaccaaaacttttatgcctattttgctgtatgcaatgtcctccattgaactacttggtatgaaccatgttttcttgatcaatcccagattagtatactttttcatctacttctagctgaggatcttccagctgaatttgaactgaataaagacaagataatgatagtccaaaaataagtattttccctatgatccagccttgacttcttttccatgacactctctttggttttcgttattctttatattcctctcttcttgactcttttcttaggttttcctcacttattaggattgcttttgctcatttattactcatttgtttgttcatttgttgccatatcccatgctcttccattattggggttatgacttttttataccatttctagcacacatttgccaagatttttcaggctgtcttacaaaaaaggttaaaatgcagactttgtatattcatttttccatagtagatgtcaattttattttgagttcaaatatatttaatgtggaaaatacaactcttaacttccatagcattccctacaaaattgttagctacagctgtcagcaaatgctcaagaattcaagagtggcttttagatctctgctaaaagggtggcctttattaacctccttggcttttggcttttgtaaagaaaagatgtacttccaatgaatcttgattcgtcacacttgcacagagtcatacacacgcacactacaaaaatacagcaaaacaattttttctacttttccttttgcattattaaatcattgtctctgcctggtgtttatttaaccacttttggcatatctacatattagttatagaaacatgtaacacatcctgccttttaagggatgtttatcttttcttctttctttcccgataaataatgaactttttgaataccttaatcccacttgagtgtctgacaatccttgcataaaatttgcatacagtaaatgtgcaataattgttggttaaatagaaataaatataaaaacacttaaatgtaaaatgtttaaaatcagaagtaactccttctcattttggaatactgttacctattcttttttttaatccatcttcagtagataatgctattagggctctgaaagtaacaaatactttcataatattttgcttgaaaaatgcatgacccagattccataagattatgatggaaatatcttccagtgcaaaacttgacgcatggcaggagatcaacaagtgaatgctggttgaaaaaatgaattgccagtccctgagactgtctcatttgttttcataacttctactttattcatatttttatccctttgaactcttagcaccaagtattttctcagtatgcttttacaaattgaatgaatgagataactttcacacgttgattctcacctactttgattgctttggagtctaaaccaagtttcactggtggaagaatggaaaatattataccattacctaggaagtgttatgtggatcctatctttgagtcatgaattttacagctggacttttatcatgtcattagtagatggctcatgatctaattcctccttggagcacaggtagaggcccagtacactccacagttatatacctcctatatatagtttattgtgagtatttgaaggcagaaatgtcatcctgttcatctttgtgctttaaaggttcaagtcccattacattgcaccactgctactctggccctctgaacatttataagagtcataggtatatagttgatatataataaatgtttactaaatgaataatttaataaaataattgactcatgcaattgccctgaaaagcaggggaatgaagcagccagcagtacttccaggaagcaactgagaaacaccctttggcacccaccattcaatcaacttaacatcagccttgaagcttaagaaataaaattaagggccaagtaagcatagctcaagccaactacttttgcattccccaggccactgattgcctcactaaatctctcactttgctgaatatgtgaaatcaattattattataggatacaaaagacagaaactttttggaaaattgttttgattattaataaaagatgacaatgtcaaggcatgagttaccaccataaaactccacgttagcattatttgaattcagtagaacaaagacaagtagatttgtcaaattacaaccctgattgtgtctcctatgcatctccattacccaaaccagtgtaatgttctcaatagttgctttcttccaataggggagcactgtaaggtcttataaaaatacctaactttcaggttcccacagaaactgagggaatcaccccagatctaacctcccactcacctttgtcaacttcactgccattttatttctttgatttctgcccagtttcttggcctctaacttcggtcctccagaccagaaacctttgaagcactgtgcagactatctgttcttgactctgtgccaaccttggatattgccttaaagatttgcctgacttgatttccagcttgagaattagcatagctctacttttatcgtggtgcccatctctggtcctttagaagctcaaacttcaaccaacaaaggcccaggacccttttgcttccccaagaggaatgaatgttcaaacagaatcatctccttctgtaaacagtgatctgtgtccttttcctcaccctctttccccatttaaaacctctttgtttgcatacttcatttgtccagttcttactgacagttccagactatctcagccatcaactcaggaagctatactaaccaatttgtaactgaacatctcaagcataccttgatatgcctctcattttccccttatctcctcctgcctcatatttgaataatcttttaaggtataaatatgttatattaactctgttaatgactgtgattatttttgagagctctaggggagctatattatataattcttggtatctctcactgtacttgcagagtgctttgcacacaagtaaaccacagaatggtatacaaatgaatatttaaatgtttagctgtttttgaccaattattcttaatattggcttcttatgacttattaccttgtgaaatattttgttagttgcatatctagaaaggttgataaattcagatcagctccaccattcttgtgtaattattattattttctaaatgttcaataatttaaatagtacttatgtacaattaattcttgatttgatctatggcataggtctgtgcccaggtgcttgggaaattgactttgacatggaggtttggatgcaggcggctaatagggaaataaactaggcaatagcaccagtgctagtgtgaaaaacaaaggattcgacagaaaaagttgaactccaatgcaagtgaaaatgaggccttggggcacctgggtggctcaggttgtgattctggggtcctgggatcgagtcctacaacagactccctgcagggagcctgcctctccctctgcttatgtctctgcttctccttctgtgtctctcatgaataaataaataaaatcttaaaataaataaaataaaaaatgggggggggagggaatggagcagccataaggactagttatatgagtgttggttttctcaaaagcatacttggagaaaatgttaactttttaatgatagatttaatatattaatatatgccaagtacttagtgtaatgcttatcatgtggtcactgctcgctgaattgaagttgcctgaatctagcttggaggcaggtgtgagtaagtcataaattaactatgaaatgacctttatggctttagcaactctataagatcccatagtgaccacattcattattaagtaatctggtcttaatcttgataactgattttgaatttatgactcctcttttactccctcatctacatctcttcaactgcattgttaatcttttcagtcctgtgcgtgtcatatcaacttcctcccgttaccttgtctcatgatgttctactctattttcttccagaaagtgaactgtccctcctctctataaaatcacatcttaaatgtcctctctcccacttacataaggcttccttatgcaaggattactttgttttttagacttctttacctcctccgaagtcttgcacttattaatatgtattactcttttataaatcaggattttggagattaatttaaatacagtaaaattcaccttttgagtttgccaaatggcaaactgtcattcaaccaaactaccattaagttgtagaacatttcaactaccatgaaaagtttcctcataactatttgtggtcagtcccatcctcctccaccccccatacctggtgaccactagcggagtttctgtcttcacagttttgcctttcccagaatttcatttaaacagagtcatataatatgtgttttctggcataatgcttttgaggtccattcattgtttcatgtatcaataggtcattccttctaatggctaagtagtattccattacatggatataccacaatttgtttatatatttactacgttgatgggcctctggcttgatttcaatatttaactattatgaataagtttatgtaaatattaatatacagatctttgtgtggaaaatgtttcaattttttcccattaataatgctgagggaaactgctggattatgaggtataattgtttaacattacttgaaaattccaacttctttttgtacatatttgtactgttttgagttataaaaaactgtgtttctgtttaatcacaaactaattaaactgggtaaatatttttttaactttagatacctacttgctatatagtaacacgtcggtgtggtcttaattcggattttactaatgactaatgatgttgagcattgcttccagccagctatgtatctcctttggtggagtgtctgtgtaaatcttttgcccattgaaaaaactgggttattttttcttcttcttgattcctatgagtattcatatgttctaaatatgcctctatcaaacaggtattttgaccatattttctcttcatctgtagcttgtctttacattctctaacagtccctttgtgaagcagattttatttatttatttatttatttatttatttatttatttattatttttttaaattttcatgagagacacacagagggagaggcagagacaggcagagggagagaagcaggcttcatgccaggagcctgatgtgggcctcaatcccgggtctctgggatcatgccctgggccgaaggcaggcgctaaactgctgagctacccagggatccccctgtgaagcagattttaatgttgattaagtccagttatttttttctaggtttcctttgtgtcctatttaaaatatctttgtctaatcaaaagtcacaaagttgacaaagtcctcctgttttttctcctggaagttgtacagttttaatttttatatttttaagtctgtgatacatttaatttcttctataaggtgtaaggtaagggctgaggctcatattttgcatgtagatgtgccattgtttcagcactctttgctgaaaagactacttttcccttcaaattacctgggaagttttttgtaaatcaacagaatatatacgtatggatgtatacctttcattatggattagtgaaaagatcatgattttggggttatactaacatttaaatcctgtctctatcattttctatgttttctaaggccatttattatgaatcagttccttaatctgaaaaaataattaaaatataaaatttttgaggtttaataactgtaaaataagcaagacatattaaatttttagtgtccaggcagtactcaaaagtggtatttgtgtttacttgtttttttttgttgttgttgttttttttttttttacttgtttttaacaattttcaaatcttccctttctagttagatgagtagtctcttaagtcagggactgttttgttttgttttgtttttttctaattctacaattacagccacaatcttcagggtagatattgtgtcctatggaggataagatactgagggtggtttgaattttcaaagaagatgatgaagaccatgaccactggctgtgtaagcaaattattaaattaatcaaagttcagaggagatcaagaatgttgggctatctctatgctccttaatcatgtacatactgctttgttttatcctctctctgttcaataggacatgaaattcaacataaatcctactgatttagaccaaagatcaatagcattttattcactataaattgcttcattattttgcagggtctccattctcataacctaaaattaaccgtttctttaaaattgcccaaattccaaagtataagccaaacaaaattgttgattgctttgggaagggttttatgaagttaagaggagcagaaatacattgctttcaaggaatttgttatttaaaatgaatgcaaaaatgtttgcagaatagattgttgaactgggagtcaaattagtttggttcaaattccactctgcctttgaataaaaagcaagttgttgaactgccttaactgccaactttattatccataaataggagctaattcacctcaaaaggcctttgcatgcacgaatgagataataaatgtgaaagcactttgtaatcactaacatcctatacaaataaaagtcattgttattacgatgattcaaagccaactctagaaagaatatgttatttgttgatcaatgaaaatgacccctggagacctccttcccctagtcaaggaacactctactaagatgtaatactacctgtcatttcaccatgtgcactgcattgctttatgttaagtaagccctctcagatttccttttaaaatggcaatacctcctaaagagtaataattaaagtcacagctcagagtgcataggtctcaaggactttctgttaaatacctggcacctgagcaacttttacatgtgtcttttcagcagctctgactagggcagcaaaacataaaaacatgagtcctagaagacttgaaaagagtaaatggggaaaaaaaaccaacctagaaactagagaatatgttagagagaggtagaaaagaacaagagtctctttccaattgtccagggtaagccttgtaaatagcagacattaaagatatattgcactgataattataaccagtatgtattttttaaattactgaggtataattgacataaataattgcttgtatttaagtggacaatgtgatgaattggggcctatgtagatgcccatgaaaccattaccacagtcaagaaaatgaacaaaaccctcaaccctctgcctactgcaaatgtcatccctctggttcctcccattttcccttccatggcctacgtccatcattccgtagtactttgaatttcttagcattttatgaaaatagaatcatatgatatatattctttaaaaaatctggttcctttttaaaaaagattttatttatttgagagagagaaagagagtgcacaagtggagagaggagccaagggagagggagaagcagactccccactgagcaggaagcttgatgggagggctcaatcccaggacctggagaccatgacctgagctgaaggcagacatttaaccaactgagccacccaggcacccaaatctggttcctttcactctgcacagttattttgaggttgatccaggttatactatgtatgaagttaattcctttttattgctgaatagtattccattgtatgactatcagtttgttcttctacatgttgatggatgtttggatgatgccattgtttaacttttatgaataaagcttctatgaacacaagcatacaactctttctatagacacttgctttcatttatcaggtaactatccaggagcaagatagctgaattatatgtcaagtatatttttaattttgtaagaactgttttccaaagtggttacaccattttgcttgaccaccagcagttcattggagttccaattcctccatatttctccaatacttggtatggccagtgtttttttgtcttaaccattctaataggcatgtagggatatcactatggtttgatttacatttcattaatgactaataatatttagcattatttgatgtgcttatttgccaactatgtaaatcttctgtggtgaaatgactttaaatttttacctttttgttggtttgttttatcttcacatcattggactttgaaaattccttattttgagtacaagtctattatcagttaaatgcttttcaaatattttccccagtctgtgacgtatcttttccttctctttgtagactgttgaatctatttctggactctattctgttcattgacctatttctctatctttacacaaataccacatttttattactatagccttaaaataagtctagaaatcggtagtatcagtcctccaacatattcctttctttcaaagtgagtatggctatcctagattatctttccctaagtgttttaaatctgtttgttaattcaacaacaggaggaggaggaagagaattctggtggagttttggttgtcgtttctttgaatatttagatcaatttgggggagaagtgacataatgttgaatcttccaatctaactatttctgagcaataagtaaggcagtatgctatcgtttagttttcttgtttcttgtgcttgaggttcattgagtttcttatatctgtgagttaataatttccatcaaatttggaaaatttgcaggcattattccttcaaatacattttctcctctctccttcaagggatccagttttatgtatattaaaccatttgaaattgtcccactacttgctg

SEQ ID NO: 2 - DNA, 20 bp, Artificial
  Synthetic primer
ccttcccttgggagattagc

SEQ ID NO: 3 - DNA, 20 bp, Artificial
  Synthetic primer
aatgatgtgtttgggggaaa

SEQ ID NO: 4 - DNA, 20 bp, Artificial
  Synthetic primer
ggtgacgtctggattggatt

SEQ ID NO: 5 - DNA, 20 bp, Artificial
  Synthetic primer
acttccccactttgcctacc

SEQ ID NO: 6 - DNA, 20 bp, Artificial
  Synthetic primer
caaatgtgctgatggaaacg

SEQ ID NO: 7 - DNA, 20 bp, Artificial
  Synthetic primer
aactgcagccacatgtgaag

SEQ ID NO: 8 - DNA, 20 bp, Artificial
  Synthetic primer
gcagggcatttttgaggtag

SEQ ID NO: 9 - DNA, 20 bp, Artificial
  Synthetic primer
gaatagccctgtggggtgta

SEQ ID NO: 10 - DNA, 20 bp, Artificial
  Synthetic primer
tcactcctgctcacatctgg

SEQ ID NO: 11 - DNA, 20 bp, Artificial
  Synthetic primer
tctcgacagacaccgtttca

SEQ ID NO: 12 - DNA, 20 bp, Artificial
  Synthetic primer
aacggaagcactgaatcaaa

SEQ ID NO: 13 - DNA, 20 bp, Artificial
  Synthetic primer
gcaaatccaagccctattca

SEQ ID NO: 14 - DNA, 25 bp, Artificial
  Synthetic primer
gctcagagtgcataggtctcaagga

SEQ ID NO: 15 - DNA, 26 bp, Artificial
  Synthetic primer
ttttggggttttgttgctattgttgt

SEQ ID NO: 16 - DNA, 26 bp, Artificial
  Synthetic primer
tgctgtgttaaaaagctatccttgga

SEQ ID NO: 17 - DNA, 30 bp, Artificial
  Synthetic primer
ttcatttacaatgcctagggatttttagga

SEQ ID NO: 18 - DNA, 24 bp, Artificial
  Synthetic primer
tcttctgagttctcttcccacaca

SEQ ID NO: 19 - DNA, 22 bp, Artificial
  Synthetic primer
gaggcaactggagttcctatgg

SEQ ID NO: 20 - DNA, 20 bp, Artificial
  Synthetic primer
tgcagggtctccattctcat

SEQ ID NO: 21 - DNA, 20 bp, Artificial
  Synthetic primer
gcttaccctggacaattgga

SEQ ID NO: 22 - DNA, 20 bp, Artificial
  Synthetic primer
tgcagggtctccattctcat

SEQ ID NO: 23 - DNA, 20 bp, Artificial
  Synthetic primer
gcttaccctggacaattgga

SEQ ID NO: 24 - DNA, 7 bp, Canis familiaris
tgaggag

(19) United States
(12) Patent Application Publication
(10) Pub. No.: US 2012/0021928 A1
(43) Pub. Date: Jan. 26, 2012

(54) GENETIC RISK ASSESSMENT FOR SHAR-PEI FEVER

(76) Inventors: Kerstin Lindblad-Toh, Malden, MA (US); Mia Olsson, Knivsta (SE); Linda Tintle, Wurtsboro, NY (US)

(21) Appl. No.: 13/161,213

(22) Filed: Jun. 15, 2011

Related U.S. Application Data
(60) Provisional application No. 61/356,230, filed on Jun. 18, 2010.

(51) Int. Cl. C40B 30/00; C12Q 1/68; C07H 21/04

(52) U.S. Cl. 506/7; 536/24.31; 536/24.33; 435/6.12

(57) ABSTRACT

The present invention provides genetic tests for the copy number or a duplication of an upstream element of the Hyaluronic Acid Synthase 2 (HAS2) gene on chromosome 13 in Shar-Pei dogs to assess risk for a Shar-Pei dog to develop Shar-Pei fever. As Shar-Pei fever is a serious disease, and its chronic state involves a high risk of developing systemic amyloidosis and kidney failure, a genetic test for the copy number of the duplication will be useful in Shar-Pei breeding. Also provided are probes and primer pairs for use in these methods.

This application claims benefit of priority to U.S. Provisional Application Ser. No. 61/356,230, filed Jun. 18, 2010, the entire contents of which are hereby incorporated by reference.

### BACKGROUND OF THE INVENTION

I. Field of the Invention

The present invention relates to the field of genetics and molecular biology. More specifically, the present invention relates to methods for risk assessment of the development of Familial Shar-Pei fever, an auto-inflammatory disease common in the Shar-Pei dog breed.

II. Related Art

The Shar-Pei dog has been a companion animal within China for centuries commissioned to guard and hunt, and sometimes serving as a fighting dog. At the beginning of the communist era, dog ownership was associated with very high taxes and the breed was close to extinction when a few specimens were exported to the United States in the early 1970's. Shar-Pei descending from this handful of dogs have undergone strong selection for their wrinkled skin phenotype and are called the “meatmouth” type. An ancestral Shar-Pei referred to as the “bonemouth” type still occurs and it presents with a less accentuated skin condition.

The major constituent of the deposit in the thickened skin is hyaluronic acid (HA), a multifunctional, linear, high-molecular-mass glycosaminoglycan, widely spread throughout epithelial, connective and neural tissues (Fraser et al., 1997). The biological role of HA depends on its size, location and equilibrium between synthesis and degradation (Laurent and Fraser, 1992; Fraser et al., 1997). Shar-Pei show two- to five-fold higher serum levels of HA compared to other breeds (Zanna et al., 2008), which has proposed the term “hyaluronanosis,” also used for a comparable human condition (Ramsden et al., 2000). The concentration of serum HA varies with age, between days, and with time of day as well as with sampling occasion (Lindqvist and Laurent, 1992), suggesting that serum HA is an unstable measurement potentially influenced by random release of HA from other tissues. HA is synthesized by three enzymes, Hyaluronic Acid Synthase (HAS) 1, 2 and 3 (Itano and Kimata, 2002; Weigel et al., 1997), with HAS2 being the rate limiting-enzyme (Weigel et al., 1997). HAS2 is overexpressed in dermal fibroblasts of Shar-Pei compared to other canine breeds (Zanna et al., 2009) suggesting a regulatory mutation as causative for hyaluronanosis. HA is deposited in certain areas of the skin of Shar-Pei, often around the head and as “socks” around the hocks. Almost all Shar-Pei seem to be affected by hyaluronanosis, with the extent varying among individuals. However, most individuals show less skin folds and hyaluronanosis with age (Ramsden et al., 2000).

Shar-Pei also suffer a strong predisposition for an auto-inflammatory disease, Familial Shar-Pei Fever (FSF), that clinically resembles some hereditary periodic fever syndromes in humans, such as Familial Mediterranean Fever (FMF) (Rivas et al., 1992). Both diseases are auto-inflammatory, characterized by seemingly unprovoked episodes of inflammation. It presents in both FMF and FSF as short (12-72 hours) recurrent bouts of high fever, accompanied by localized inflammation usually involving the joints (especially the ankles in humans and the corresponding hock joint in dogs).

Patients with FMF and Shar-Pei with FSF seem free of symptoms between episodes that can occur as often as every few weeks. However, since acute phase reactants endure between episodes, a subclinical state and chronic auto-inflammation might persist. The chronic state puts the patients at risk of developing reactive systemic amyloidosis and kidney or liver failure following the accumulation of acute phase proteins in the organs. In Shar-Pei, the episodes are typically more frequent during the first years of life and the frequency of Shar-Pei dogs with unexplained fever episodes was estimated to be 23% in the United States in 1992 (Rivas et al., 1992).

Up to this point it has been unknown why Shar-Pei dogs are predisposed to FSF and there are currently no methods or genetic tests that can predict whether a Shar-Pei dog will develop FSF and the serious consequences of reactive amyloidosis and, e.g., kidney failure. Such methods and tests would be of great value for breeders of Shar-Pei in their selection of breeder animals and would thus reduce the risk that future generations of Shar-Pei will develop FSF.

### DESCRIPTION OF THE INVENTION

The present invention provides methods for testing a Shar-Pei dog for its genetic predisposition to develop Familial Shar-Pei fever, the method comprising analyzing a nucleic acid sample obtained from said canine for a regulatory mutation in the Shar-Pei genome.

The present inventors have discovered a regulatory mutation, a 16.1 Kb duplication (SEQ ID NO:1), upstream of the Hyaluronic Acid Synthase 2 (HAS2) gene on chromosome 13 in Shar-Pei dogs. A high copy number of the duplication, present only in Shar-Pei, is associated with severe fever. Accordingly, genetic tests for the copy number of the duplication allow the assessment of the risk for a Shar-Pei dog to develop Shar-Pei fever. A high copy number (>10) of the duplication constitute a high risk for Shar-Pei fever, a low copy number (<5) a low risk, and a copy number between 5-10, an intermediary risk. As Shar-Pei fever is a serious disease, and its chronic state involves a high risk of developing systemic amyloidosis and kidney failure, a genetic test for the copy number of the duplication will be useful in Shar-Pei breeding.

One aspect of the present invention provides a method for testing a Shar-Pei dog for its genetic predisposition to develop Familial Shar-Pei fever method comprising obtaining sequence information from a nucleic acid located upstream of the Hyaluronic Acid Synthase 2 (HAS2) gene on the canine chromosome 13 to determine the number of duplications present in this region, wherein a higher number of duplications is indicative of the risk that the dog will develop Familial Shar-Pei fever.

The region of nucleic acid located upstream of the Hyaluronic Acid Synthase 2 (HAS2) gene on the canine chromosome 13 may comprise the nucleic acid sequence consisting of bp 22,937,592 to bp 24,414,650 (CanFam 2.0 Chr13: 22,937,592-24,414,650).

Obtaining sequence information can comprise sequencing a portion of the region of nucleic acid located on chromosome 13 CanFam 2.0 Chr13: 22,937,592-24,414,650 to determine the number of duplications present in the region. More particularly, the duplication can consist of the nucleic sequence SEQ II) NO:1 or sequence having more than 95% identity to SEQ ID NO:1, such as more than 99% identity to SEQ ID NO:1.

Obtaining sequence information can also comprise using a nucleic acid probe that hybridizes to a part of the nucleic acid sequence SEQ ID NO:1, or a sequence complementary thereto, under stringent conditions.

Obtaining sequence information can comprise using a nucleic acid primer pair that amplifyies a nucleic acid comprising a part of the duplication to determine the number of duplications present. The nucleic acid primers can be selected from SEQ ID NO:2, SEQ ID NO:3, SEQ ID NO:4, SEQ II) NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ II) NO:14, SEQ ID NO:15, SEQ II) NO:16, SEQ ID NO:17, SEQ ID NO:18, SEQ II) NO:19, SEQ ID NO:20, SEQ ID NO:21, SEQ ID NO:22, and SEQ ID NO:23.

For those skilled in the art, there are many techniques available to analyze nucleic acids and to determine the copy number of a genetic element. Some of the techniques that can be used according to the present invention include; Real time PCR, Southern blot analysis, DNA sequencing and DNA array analysis.

In another aspect, the present invention provides an isolated nucleic acid probe comprising a sequence present in a duplication on the canine chromosome 13 corresponding to the nucleic acid sequence SEQ ID NO:1, and wherein the nucleic acid probe hybridizies to a part of the nucleic acid sequence SEQ ID NO:1, or a sequence complementary thereto, under stringent conditions.

In another aspect, the present invention provides an isolated nucleic acid primer pair comprising a first primer and a second primer, wherein each of the first and second primers comprises a sequence present in a duplication on the canine chromosome 13 corresponding to the nucleic acid sequence SEQ II) NO:1, and wherein the primer pair amplifyies a nucleic acid containing at least a part of said duplication.

In another aspect, the present invention provides an isolated nucleic acid primer comprising a sequence present in a duplication on the canine chromosome 13 corresponding to the nucleic acid sequence SEQ ID NO:1.

In another aspect, the present invention provides an isolated nucleic acid primer pair comprising a first primer and a second primer, wherein each of the first and second primers comprises a sequence present in a duplication on the canine chromosome 13 corresponding to the nucleic acid sequence SEQ ID NO:1, and wherein the primer pair amplifyies a nucleic acid containing a breakpoint of the duplication.

It is contemplated that any method or composition described herein can be implemented with respect to any other method or composition described herein. These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

The use of the word “a” or “an” in the claims and/or the specification may mean “one,” but it is also consistent with the meaning of “one or more,” “at least one,” and “one or more than one.”

The phrase “one or more” as found in the claims and/or the specification is defined as 1, 2, 3, 4, 5, 6, 7, 8, 9, 10, or more.

Throughout this application, the terms “about” and “approximately” indicate that a value includes the inherent variation of error for the device, the method being employed to determine the value, or the variation that exists among the study subjects. In one non-limiting embodiment the terms are defined to be within 10%, preferably within 5%, more preferably within 1%, and most preferably within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

### BRIEF DESCRIPTION OF THE DRAWINGS

The invention may be better understood by reference to one or more of these drawings in combination with the detailed description of specific embodiments presented herein.

FIGS. 1A-C. The breed-specific disease association and the strongest selective sweep signal in this breed are present in the same region on chromosome 13. (FIG. 1A) A 10-fold reduction of heterozygosity was identified on chromosome 13 when comparing Shar-Pei (n=50) to 24 other canine breeds (n=230). 50,000 SNPs were used to screen the whole genome using a sliding window approach (see methods). (FIG. 1B) A case-control GWAs analysis identified five SNPs in a 2.7 Mb region on chromosome 13 (CanFam 2.0 Chrl3: 27,9-30,7 Mb) significantly associated with Familial Shar-Pei Fever (FSF), after correcting for multiple testing using 100,000 permutations. The Shar-Pei dogs (n=40) used in the study were strictly classified into groups of affected (n=23) and unaffected (n=17) by FSF. (FIG. 1C) SNPs associated with FSF are interspersed with the signals of selection. Individual SNPs either show a signal of association (black line) or show a high rate of homozygosity (grey line).

FIGS. 2A-C. A de novo duplication with high copy number upstream of Hyaluronic Acid Synthase 2 (HAS2) in Shar-Pei. (FIG. 2A) Targeted resequencing of a 1.5 Mb region on chromosome 13 identified a duplication (CanFam2.0 Chr13: 23,746,089-23,762,189) with on average 3.5-4.5× higher read coverage in two Shar-Pei (black and grey curves) compared to three control breeds (grey—Standard Poodle, grey—Neapolitan Mastiff and grey—pug). (FIG. 2B) The duplication was determined to be 16.1 Kb long with both breakpoints located in repeats (a SINE and a LINE) with an insertion of 7 bp separating different copies. (FIG. 2C) Southern blot analysis of control breeds, bonemouth and meatmouth Shar-Pei showed different fragment length for the duplication including both healthy (h, not affected by fever) and fever (f) affected Shar-Pei. Vertical lines in copy 1 and copy 2 indicate the restriction sites of BsrGI. The estimated fragment sizes are given to the right.

FIG. 3. Copy number estimates in eleven control breeds and Shar-Pei. All dogs (control, n=27; Shar-Pei, n=62) were analyzed in triplicate and the range (average+/−SD) plotted. Control dogs have average of two copies (one per chromosome), whereas all Shar-Pei dogs carried the duplication and demonstrated copy number ranging from four to eighteen.

FIG. 4. A centromeric probe on the BsrGI Southern suggests bonemouth Shar-pei harbor a partial duplication. This Southern blot shows a smaller hand in the bonemouth type in using a centromeric portion, whereas the probe in the telomeric portion of the duplication does not. This indicates that bonemouth Shar-Pei carry a partial duplication with a different breakpoint. One meatmouth Shar-Pei (known to have several bonemouth Shar-Pei in its pedigree) seemed to be heterozygous for the different copies.

FIG. 5. Relation between serum hyaluronic acid (HA) concentration and copy number. None of the control dogs (grey) have the duplication whereas it is present in all Shar-Pei dogs (black, using centromeric probe). All dogs with HA levels higher than 600 μg L⁻¹ carried the duplication although no correlation could be found between HA concentration and copy number.

FIG. 6. A higher copy number of the identified duplication correlates with Familial Shar-Pei Fever. A significant correlation (P=0.0006) was seen using an unpaired t-test comparing copy number in unaffected individuals (n=15) and individuals severely affected by FSF (n=10). Unaffected dogs had no history of unexplained fever and inflammation and had no relatives with a history of recurrent fevers or amyloidosis. Severely affected dogs were all diagnosed with recurrent fever and amyloidosis at post-mortem examination. Based on this limited sample size, most dogs with more than ten copies had fever whereas most dogs with <6 copies did not.

### DETAILED DESCRIPTION OF THE INVENTION

### A. Methodology

Any appropriate method can be used to determine the number of duplications present in the region of nucleic acid located upstream of the Hyaluronic Acid Synthase 2 (HAS2) gene on the canine chromosome 13 of a Shar-Pei dog. For example, the number of duplications can be determined by nucleic acid sequencing, denaturing high performance liquid chromatography (DHPLC; Underhill et al., 1997), allele-specific hybridization (Stoneking et al., 1991; and Prince et al., 2001), allele-specific restriction digests, polymorphism specific polymerase chain reactions, single-stranded conformational polymorphism detection (Schafer et al., 1998), infrared matrix-assisted laser desorption/ionization mass spectrometry (WO 99/57318), and combinations of such methods.

Genomic DNA can be used to determine the number of duplications present in the region of nucleic acid located upstream of the Hyaluronic Acid Synthase 2 (HAS2) gene on the canine chromosome 13 of a Shar-Pei dog. Genomic DNA can be extracted from a biological sample such as peripheral blood samples, hair roots, or tissues (e.g., mucosal scrapings of the lining of the mouth or from renal or hepatic tissue). Any appropriate method can be used to extract genomic DNA from a blood or tissue sample, including, for example, phenol extraction. In some cases, genomic DNA can be extracted with kits such as the QIAamp® Tissue Kit (Qiagen, Chatsworth, Calif.), the Wizard® Genomic DNA purification kit (Promega, Madison, Wis.), the Puregene DNA Isolation System (Gentra Systems, Minneapolis, Minn.), or the A.S.A.P.3 Genomic DNA isolation kit (Boehringer Mannheim, Indianapolis, Ind.).

Amplification methods such as PCR techniques can be used to determine the number of duplications present in region of nucleic acid located upstream of the Hyaluronic Acid Synthase 2 (HAS2) gene on the canine chromosome 13 of a Shar-Pei dog. For example, a primer pair designed to amplify PCR products containing a duplication breakpoint can be used to determine the number of duplications. Such a primer pair can contain a first primer that anneals upstream of the duplication breakpoint such that extension from that primer proceeds toward the duplication breakpoint and a second primer that anneals downstream of the duplication breakpoint such that extension from that primer also proceeds toward the duplication breakpoint. When a sample contains nucleic acid with the duplication, an appropriately sized PCR product containing the duplication breakpoint can be generated and detected.

The present invention also provides kits that can be used to determine the number of duplications in the region of nucleic acid located upstream of the Hyaluronic Acid Synthase 2 (HASP) gene on the canine chromosome 13 of a Shar-Pei dog. Such kits can include nucleic acid molecules (e.g., primer pairs or probes), control nucleic acid molecules (e.g., nucleic acid comprising the duplication or apart thereof), DNA aptamers, microarrays, or data analysis software optionally together with any other appropriate reagents, tools, or instructions for performing the methods described herein. Appropriate informational material can be descriptive, instructional, marketing, or other materials that relate to the methods described herein or the use of the reagents for the methods described herein. For example, the informational material can relate to performing a genetic analysis on a Shar-Pei dog and subsequently classifying the horse as being at risk (or not) for developing melanomas. In addition, or in an alternative, the informational material of a kit can be contact information, for example, a physical address, email address, website, or telephone number, where a user of the kit can obtain substantive information about performing a genetic analysis and interpreting the results.

Hybridization may particularly be performed under stringent or highly stringent conditions. “Stringent or highly stringent conditions” of hybridization are well known to or can be established by the person skilled in the art according to conventional protocols. Appropriate stringent conditions for each sequence may be established on the basis of well-known parameters such as temperature, composition of the nucleic acid molecules, salt conditions, etc. See, for example, Sambrook et al., “Molecular Cloning, A Laboratory Manual,” CSH Press, Cold Spring Harbor, 1989 or Higgins and Hames (eds.), “Nucleic acid hybridization, a practical approach,” IRL Press, Oxford 1985, see in particular the chapter “Hybridization Strategy” by Britten & Davidson. Typical (highly stringent) conditions comprise hybridization at 65° C. in 0.5×SSC and 0.1% SDS or hybridization at 42° C. in 50% formamide, 4×SSC and 0.1% SDS. Hybridization is usually followed by washing to remove unspecific signals. Washing conditions include conditions such as 65° C., 0.2×SSC and 0.1% SDS or 2×SSC and 0.1% SDS or 0.3×SSC and 0.1% SDS at 25° C.-65° C.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods

Samples and diagnostic procedure. DNA was extracted from blood samples using QIAamp DNA Blood Midi Kit (QIAGEN) or PureLink™ Genomic DNA (Invitrogen).

Shar-Pei fever. Shar-Pei dogs were classified affected or unaffected by FSF based on a strict inclusion and exclusion criteria. Shar-Pei classified as unaffected were older than 5 years with no experience of unexplained fever and/or inflammation and also had no close relatives at the grandparental level that could be classified as affected. Shar-Pei classified as affected had experienced recurrent episodes of high fever accompanied with inflammation of joints from an early age (<one-year old). Affected individuals who were subjected to post-mortem examination were sub-classified as severely affected if depositions of amyloid in kidneys and/or liver were detected (amyloidosis).

Hyaluronanosis. Serum hyaluronic acid (HA) concentration was used as a proxy for hyaluronanosis but no cut-off value was established. HA measurements were performed using the Hyaluronan ELISA kit (Echelon Biosciences INC) according to the manufacturer's instructions. The absorbence was read at 405 nm, and a semi-log standard curve was used to calculate hyaluronic acid concentrations.

Homozygosity and genome-wide association mapping. A whole genome scan was performed with two runs, 27K (v1) and 50K (v2) canine Affymetrix SNP chips. Results were called using Affymetrix's snp5-geno-qc software. The 50K array was used when the rate of heterozygosity was calculated for U.S. Shar-Pei separately and for a reference group of 24 other breeds. The ratio of heterozygosity in 10 SNP (≈1 Mb) sliding windows between the two groups was used as a measure of relative heterozygosity. To look for regions of homozygosity within the Shar Pei genome only, the software package PLINK (Purcell et al., 2007) was used to search for within individual runs of homozygosity. This was performed both for the 50 K array with 50 U.S. Shar-Peis and replicated for 37 Spanish Shar-Peis using 22,362 SNPs genotyped with the Illuninas CanineSNP20 BeadChip. These data were collected with an Illumina BeadStation scanner and genotypes were scored using GenomeStudio. Regions of homozygosity were defined if shared across all Shar-Pei samples. A case-control association analysis using 17.227 SNPs common to both the 27K and 50K arrays (n=17 227, MAF>0.05, call rate >75%) was performed in Shar-Pei classified as affected (n=23) or unaffected (n=17) by Shar-Pei fever. The software package PLINK (Purcell et al., 2007) was used for the analyses and to ensure genome-wide significance p-values were corrected for multiple testing. Values used are the max (T) empirical p-values obtained after 100,000 permutations.

Targeted resequencing. Target capture of the 1.5 Mb homozygous candidate region (CanFam 2.0 Chr13: 22,937,592-24,414,650) was performed by using a 385K custom-designed sequence capture array from Roche NimbleGen. Hybridization library preparation was performed as following: Genomic DNA (15-20 μg) was fragmented using sonication; blunting of DNA fragments using T4 DNA Polymerase, Klenow Fragment and T4 Polynucleotide Kinase; adding A-overhangs using Klenow Fragment exo⁻ and ligation of adaptors using T4 DNA Ligase with Single-read Genomic Adapter Oligo Mix (Illumina). All enzymes were purchased from Fermentas and used following manufacturers instructions. Purification steps were performed using QIAquick PCR Purification Kit (QIAGEN). Hybridization was performed following the manufacturers instructions without amplification of the fragment library prior to hybridization. Eluted captured DNA and uncaptured libraries were amplified using Phusion High Fidelity PCR Master Mix (Finnzymes) and the SYBR Green PCR Master Mix (Applied Biosystems) was used to estimate the relative fold-enrichment. Capture libraries with the estimated enrichment-factor of >200 were sequenced using Genome Analyzer (Illumina) and obtained sequences were aligned to reference dog sequence (Lindblad-Toh et al., 2005) of the targeted region using Maq assembly (Heng et al., 2008).

Polymerase Chain Reaction (PCR) and Sanger sequencing. All primers used were designed using Primer3 (Rozen and Skaletsky, 2000) and are listed in Table 2. PCRs and Sanger Sequencing were performed to investigate putative mutations (five SNPs and one indel) and were carried out with 20 ng genomic DNA using AmpliTaq Gold® DNA Polymerase (Applied Biosystems) following the manufacturers instructions. The amplification of the copy number variant (CNV) breakpoints was performed with 400 ng of DNA and a Long-range PCR with Expand Long Template PCR System Mix 1 (Roche), cloned using Zero Blunt TOPO Cloning Kit (Invitrogen) and plasmid DNA prepared using QIAprep Spin Miniprep Kit (QIAGEN). PCR products and plasmids were sequenced using capillary electrophoresis 3730×1 (Applied Biosystems), aligned and analyzed using CodonCode Aligner version 2.0.6 (CodonCode).

Southern blot analysis. Four micrograms of genomic DNA from each sample was digested with BsrGI (New England BioLabs) and separated on a 0.7% agarose gel. Hybridizations and blot visualization were performed with the Gene Images AlkPhos Direct Labelling and Detection System (GE Healthcare) following the manufactures instructions using a 910 hp probe designed to chromosome 13 (targeting CanFam 2.0 Chr13: 23,746,12-23,747,522). Hyperfilm ECL (Amersham) films were developed following exposure to the labeled blot for one hour.

Real time PCR. Relative fold-enrichment was performed using the comparative ΔC_{T}-method where an assay designed within the CNV (SP Duplication Inside) was normalized to an assay flanking the CNV (SP Duplication Outside). Fast Real-Time PCR was performed using TaqMan® Universal PCR Master Mix (Applied Biosystems) and a 7900 HT Fast Real-Time PCR system (Applied Biosystems).

### Example 2

### Identification of Candidate Loci for the Breed-Specific Phenotype Hyaluronanosis

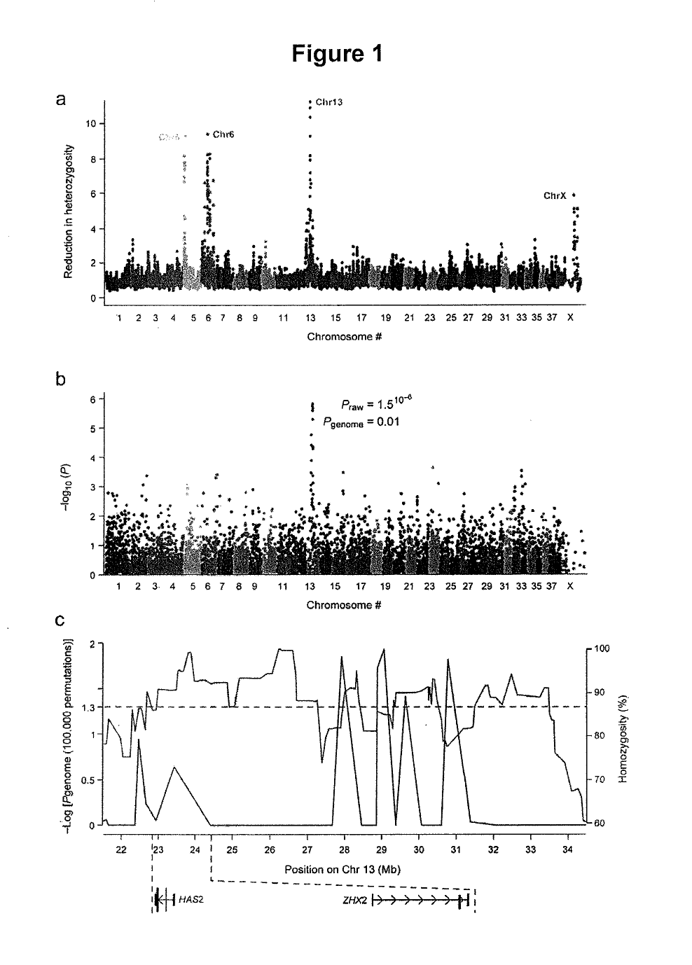

The present inventors screened the genome for signatures of selective sweeps. Selective sweeps can be recognized as long chromosomal segments with a low degree of heterozygosity within populations (Smith and haigh, 1974). Using 50,000 single nucleotide polymorphisms (SNPs) distributed throughout the dog genome the level of heterozygosity in 10 SNP windows was compared between a set of Shar-Pei (n=50, all from the US) and the average of 24 other canine breeds (n=230). On four chromosomes (Cfa 5, 6, 13 and X) the reduction in heterozygosity in Shar-Pei was greater than 4-fold the average of control breeds (FIG. 1A). The strongest signal of reduced heterozygosity appeared within a 3.7 Mb stretch on chromosome 13 (CanFam 2.0 Chr13: 23,487,992-27,227,623) near the HAS2 gene, where almost complete homozygosity was observed in Shar Pei (FIG. 1C). Here, the reduction in heterozygosity was greater than 10-fold in Shar-Pei and several smaller regions showed complete homozygosity. The same region was overwhelmingly homozygous in 37 Spanish Shar-Pei samples and was partially the same as reported in a recent study (Akey et al., 2010). The strong signal, together with the known function of HAS2, made this region an obvious candidate region for harboring the hyaluronanosis mutation.

### Example 3

### Mapping of the Susceptibility Loci for Familial Shar-Pei Fever (FSF)

The present inventors performed a genome-wide association to map the susceptibility loci for FSF, using Shar-Pei strictly classified into FSF affected (n=23) and unaffected (n=17). Interestingly, five SNPs were significantly associated (best SNP p_{raw}=1.5×10⁻⁶, p_{genome}=0.01 based on 100,000 permutations; software package Plink (Purcell et al., 2007)), all on chromosome 13 (CanFam 2.0 Chr13: 27,9-30,7 Mb, FIG. 1B). When comparing the signals of association and the regions of low heterozygosity, they appear interspersed so that individual SNPs are either part of homozygous regions or signals associated with FSF (FIG. 1C). Hence, it is difficult to determine exactly where the strongest association resides.

### Example 4

### Identification of a Duplication Upstream of the Hyaluronic Acid Synthase 2 (HAS2) Gene in Shar-Pei

Using targeted sequence capture technology, the present inventors resequenced 1.5 Mb around and upstream of HAS2 (CanFam 2.0 Chr13: 22,937,592-24,414,650) in two Shar-Pei and three control dogs from other breeds. Dogs were chosen based on HA serum levels, a proxy measurement for hyaluronanosis. The HA levels were three to four-fold higher in Shar-Pei than in control dogs (Table 1A).

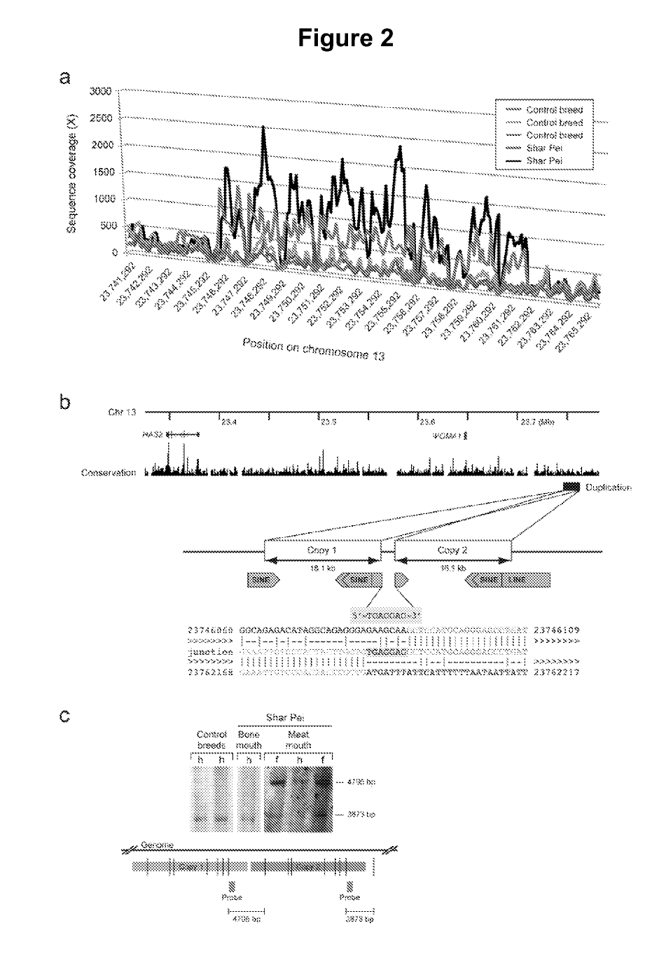

The obtained sequences were mapped to the boxer reference sequence providing at least 5× coverage for 96-98% of the resequenced region in each individual. After masking repetitive sequences we found ˜670 indels and ˜1,500 SNPs in each dog (Table 3) as well as a duplication that was present in high copy number in the two Shar-Pei (FIG. 2A).

TABLE 1A
Dogs used in resequencing and their Hyaluronic acid concentration

| | Total | |
|---|---|---|
| Breed | included | hyaluronic acid¹ |
| Shar-Pei | 2 | |
| Meatmouth type | 2 | 1484, 2052 |
| Control breeds | 3 | |
| Standard Poodle | 1 | 151 |
| Pug | 1 | 492 |
| Neapolitan Mastiff | 1 | 351 |

¹Serum concentration of hyaluronic acid expressed in μg L⁻¹

TABLE 1B
Dogs used in the Southern blot analysis

| | Number of | Affection status Familial |
|---|---|---|
| Breed | individuals | Shar-Pei Fever |
| Shar-Pei | 8 | |
| bonemouth type | 2 | Unaffected |
| meatmouth type | 2 | Unaffected |
| meatmouth type | 4 | Affected |
| Control breeds | 3 | |
| German Shepherd | 1 | Unaffected |
| Eurasian | 1 | Unaffected |
| Rough coated collie | 1 | Unaffected |

TABLE 1C
Dogs used in quantitative PCR experiments in the search of the presence
of duplication and association to hyaluronosis and Familial Shar-Pei
Fever (FSF)

| | Total | hyaluronic |
|---|---|---|
| Breed | included | acid¹ |
| Control breed | 71 | 158 |
| Shar-Pei (meatmouth type) | 56 | 831 |
| FSF² | 12 | |
| FSF + A | 10 | |
| H | 11 | |
| h | 11 | |
| Uncertain diagnosis | 12 | |
| Shar Pei (bonemouth type) | 4 | 175 |
| FSF | 0 | |
| FSF + A | 0 | |
| H | 4 | |
| h | 0 | |
| Uncertain diagnosis | 0 | |

¹Median level of hyaluronic acid expressed in μg L⁻¹
²Familial Shar-Pei Fever (FSF) status FSF, Shar-Pei reported to have experienced unexplained fever, FSF + A, Shar-Pei reported to have experienced frequent episodes of unexplained fever and positive when testing for amyloidosis, H, Shar-Pei, older than five years, reported to never have experienced unexplained feverepisodes and have no first degree relatives diagnosed with FSF or amyloidosis, h, Shar-Pei reported to never have experienced unexplained fever episodes.

TABLE 2
Primers and Probes

| | | |
|---|---|---|
| Forward primer 5′-3′ | Reverse primer 5′-3′ | TA ¹ |

Mutation screening

| | | | |
|---|---|---|---|
| Conserved | CCTTCCCTTGGGAGATTA | AATGATGTGTTTGGGGGA | 60 |
| SNP1 | GC | AA | |
| | (SEQ ID NO: 2) | (SEQ ID NO: 3 | |
| Conserved | GGTGACGTCTGGATTGG | ACTTCCCCACTTTGCCTAC | 60 |
| SNP2 | ATT | C | |
| | (SEQ ID NO: 4) | (SEQ ID NO: 5) | |
| Conserved | CAAATGTGCTGATGGAA | AACTGCAGCCACATGTGA | 60 |
| SNP3 | ACG | AG | |
| | (SEQ ID NO: 6) | (SEQ ID NO: 7) | |
| Conserved | GCAGGGCATTTTTGAGG | GAATAGCCCTGTGGGGTG | 60 |
| SNP4 | TAG | TA | |
| | (SEQ ID NO: 8) | (SEQ ID NO: 9) | |
| Conserved | TCACTCCTGCTCACATCT | TCTCGACAGACACCGTTT | 60 |
| SNP5 | GG | CA | |
| | (SEQ ID NO: 10) | (SEQ ID NO: 11) | |
| Conserved | AACGGAAGCACTGAATC | GCAAATCCAAGCCCTATT | 59 |
| Indel | AAA | CA | |
| | (SEQ ID NO: 12) | (SEQ ID NO: 13) | |

Identification of duplication breakpoint

| | | | |
|---|---|---|---|
| Duplication | GCTCAGAGTGCATAGGT | TTTTGGGGTTTTGTTGCTA | 65 |
| breakpoint | CTCAAGGA | TTGTTGT | |
| | (SEQ ID NO: 14) | (SEQ ID NO: 15) | |

Quantitative PCR

| | | |
|---|---|---|
| SP | TGCTGTGTTAAAAAGCT | TTCATTTACAATGCCTAG |
| Duplication | ATCCTTGGA | GGATTTTTAGGA |
| Inside | (SEQ ID NO: 16) | (SEQ ID NO: 17) |
| SP | TCTTCTGAGTTCTCTTCC | GAGGCAACTGGAGTTCCT |
| Duplication | CACACA | ATGG |
| Outside | (SEQ ID NO: 18) | (SEQ ID NO: 19) |

Southern Blot analysis

| | | | |
|---|---|---|---|
| Cetromeric | TGCAGGGTCTCCATTCTC | GCTTACCCTGGACAATTG | 60 |
| probe | AT | GA | |
| | (SEQ ID NO: 20) | (SEQ ID NO: 21) | |
| Teleomeric | TGCAGGGTCTCCATTCTC | GCTTACCCTGGACAATTG | 60 |
| Probe | AT | GA | |
| | (SEQ ID NO: 22) | (SEQ ID NO: 23) | |

¹ Annealing temperature in ° C.

TABLE 3
Summary statistics of the targeted resequencing compared to
full or repeat-masked genome sequence
(CanFam 2.0 Chr13: 22,937,592-24,414,650)

| | | Full Reference | | | Repeat-masked | | |
|---|---|---|---|---|---|---|---|
| | | Sequence | | | Reference Sequence | | |
| | | in Target Region | | | in Target Region | | |
| | | | | No | | | No |
| | No of | % | Cover- | of | % | Cover- | of |
| breed | reads | mapped¹ | age² | SNPs³ | mapped¹ | age² | SNPs³ |
| Nea. | 11,011,789 | 92 | 246 | 2773 | 60 | 256 | 1380 |
| Mastiff | | | | | | | |
| Pug | 9,868,613 | 74 | 178 | 3381 | 45 | 171 | 1630 |
| Poodle | 8,672,727 | 79 | 165 | 2646 | 48 | 161 | 1356 |
| Shar-Pei | 10,789,354 | 90 | 236 | 3399 | 58 | 245 | 1791 |
| A | | | | | | | |
| Shar-Pei | 9,715,905 | 75 | 176 | 3343 | 47 | 176 | 1666 |
| B | | | | | | | |

¹Proportion of reads aligned the dog reference sequence (CanFam 2.0)
²Average number of sequences covering each base position
³Number of SNPs identified in each resequenced individual compared to the reference genome (CanFam 2.0)

Six mutations (four SNPs and one indel located in conserved elements, and the duplication) were tested for correlation to the hyaluronanosis phenotype using 13 additional dogs from seven breeds. Only the duplication was concordant with phenotype, thus allowing us to exclude the other five variants. The duplication was determined to be 16.1 Kb long (CanFam 2.0 Chr13: 23,746,089-23,762,189) (SEQ ID NO: 1) with breakpoints located in repeats (a SINE at the centromcric end and a LINE at the telomeric end) and individual copies were separated by a seven base pair sequence (SEQ ID NO: 24; FIG. 2B).

### Example 5

### Confirmation of the Duplication in Shar-Pei

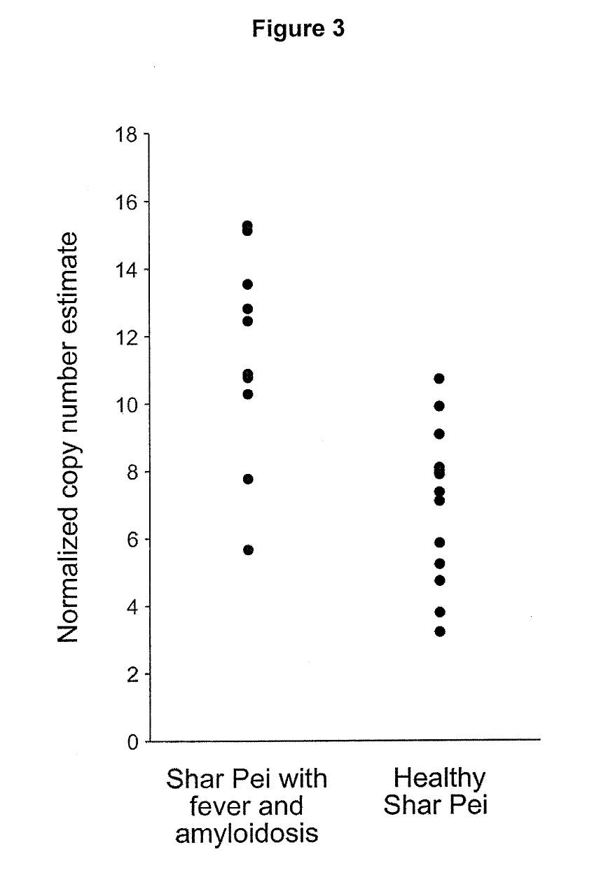

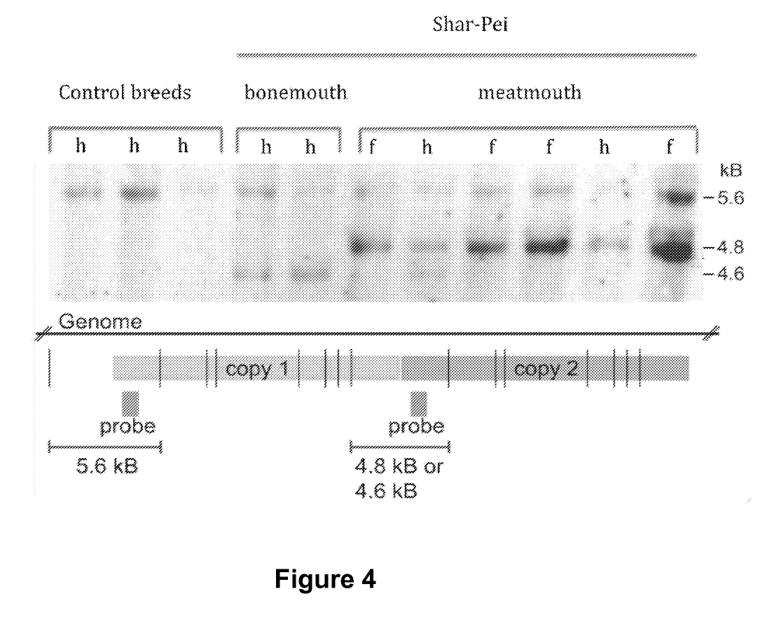

The present inventors confirmed the presence of a de novo duplication in Shar-Pei by performing Southern Blot and/or quantitative PCR in 74 dogs from 27 other breeds, none of which had the duplication (Table 1B and 1C, FIG. 3) Interestingly, with a telomeric probe on the Southern Blot (FIG. 2C), bonemouth Shar-Pei did not appear to have a duplication, but with a centromeric probe an extra band was seen (FIG. 4), suggesting that the bonemouth type might carry a partial duplication with a different breakpoint. In addition, an intensity difference was seen between different Shar-Pei (FIG. 4), suggesting variation in copy number for the duplication.

### Example 6

### Association Between High Copy Number of the Duplication and Severe Familial Shar-Pei Fever

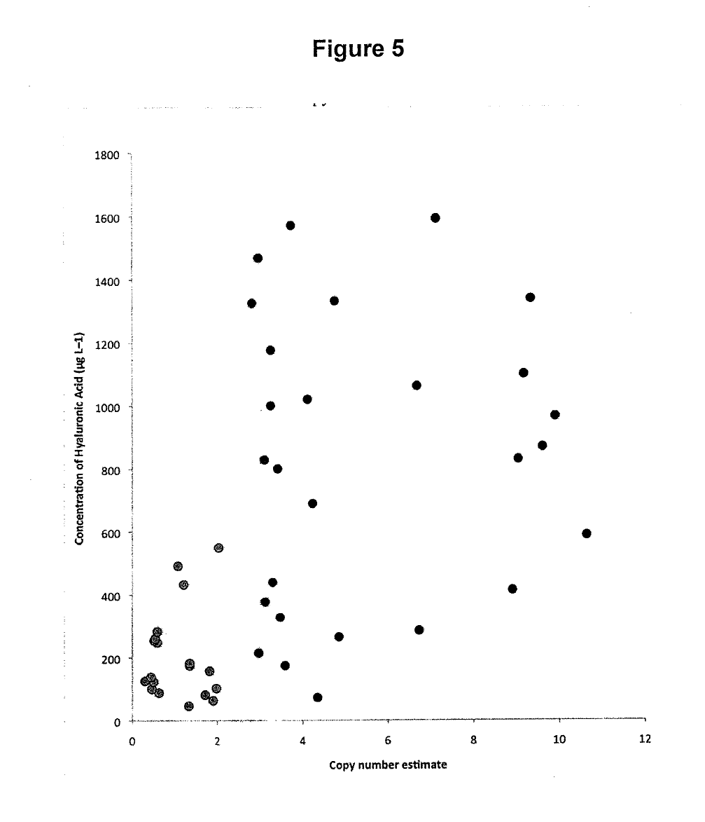

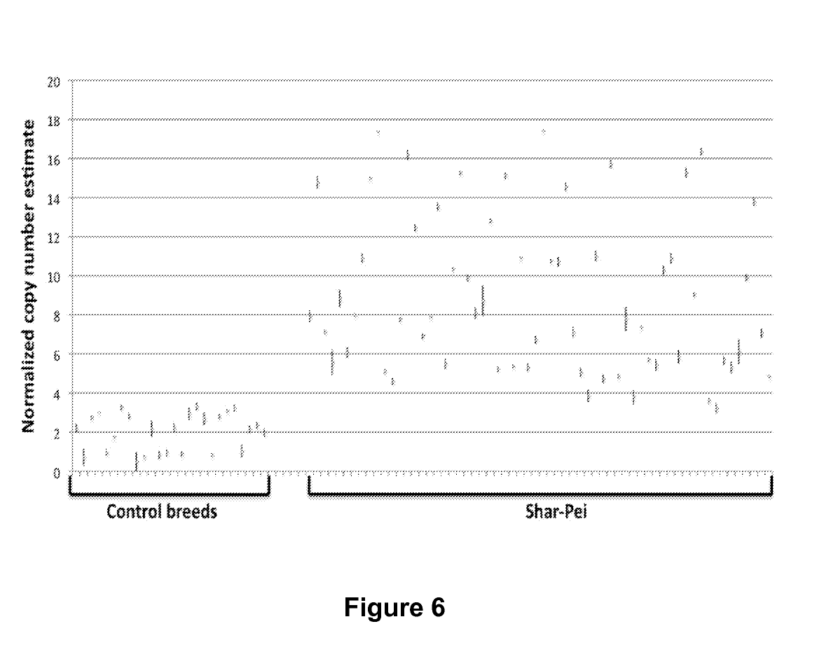

To more carefully quantify how copy number might influence the severity of hyaluronanosis and/or FSF, the present inventors performed quantitative PCR in 62 Shar-Pei, who were affected by hyaluronanosis to different degrees based on serum HA measurements and also classified as affected or unaffected by FSF. The number of copies was estimated as the relative—fold enrichment (ΔCt) between amplicons within and outside the duplication. No clear association was detected between serum level of HA in Shar-Pei and duplication copy number (FIG. 5), although the mean HA level in Shar-Pei with ≧6 copies was 905±403 μg/L, while Shar-Pei with fewer copies had an mean concentration of 770±494 μg/L and control breeds had HA serum levels of 206±145 μg/L. Interestingly, the three bonemouth Shar-Pei had between 73 and 266 μg/L, within the normal range. The difficulties to correlate serum levels of HA with other clinical and biomedical parameters have been seen in many human studies, where no or only weak correlations have been obtained (Goldberg et al., 1991; Hedin et al., 1991). Notably, the inventors obtained a significant association (P=0.0006) between higher copy number and severe FSF, in dogs that had experienced frequent episodes of fever and inflammation and had histologically confirmed amyloidosis (n=10) in contrast to Shar-Pei that were themselves unaffected by FSF and that lacked first degree relatives with fever (n=15) (FIG. 6). The observed association, despite the very high homozygosity in this region, strongly suggests that a high copy number of this duplication is not just a genetic marker for FSF but is causally related to the development of disease

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Akey et al., PNAS, 19:1160-1165, 2010.
Fraser et al., J. Inter. Med., 242:27-33. 1997.
Goldberg et al., Arthritis Rheum., 34:799-807, 1991.
Hedin et al., J. Rheumatol., 18:1601-1605, 1991.
Heng et al., Genome Res., 18:1851-1858, 2008.
Higgins and Hames, In: Nucleic acid hybridization, a practical approach, IRL Press, Oxford, 1985.
Itano and Kimata, IUBMB Life, 54:195-199, 2002.
Laurent and Fraser, FASEB J., 6:2397-2404, 1992.
Lindblad-Toh et al., Nature, 438:803-819, 2005.
Lindqvist and Laurent, Scand. J. Clin. Lab. Invest., 52:613-621, 1992.
PCT Appln. WO 99/57318
Prince et al., Genome Res., 11(1):152-162, 2001.
Purcell et al., Amer. J. Human Gene., 81:559-575, 2007.
Ramsden et al., J. Pediatrics, 36:62-68, 2000.
Rivas et al., Clin. Immun. Immunopathol., 64:36-45, 1992.
Rozen and Skaletsky, In: Bioinformatics Methods and Protocols: Methods in Molecular Biology, 365-386, Humana Press, NY, 2000
Sambrook et al., In: Molecular Cloning, A Laboratory Manual, CSH Press, Cold Spring Harbor, 1989.
Schafer el al., Nat. Biotechnol., 15:33-39, 1998.
Smith and Haigh, Genetic Res., 23:23-35, 1974.
Stoneking et al., Am. J. Hum. Genet., 48:370-382, 1991.
Underhill et al., Genome Res., 7:996-1005, 1997.
Weigel et al., J. Biol. Chem., 272:13997-4000, 1997.
Zanna et al., Veterinary Dermatol., 19:314-318, 2008.
Zanna et al., Veterinary Dermatol., 20:377-382, 2009.

What is claimed:

1. A method for testing a Shar-Pei dog for its genetic predisposition to develop Familial Shar-Pei fever comprising or consisting essentially of obtaining sequence information from the region of nucleic acid located upstream of the Hyaluronic Acid Synthase 2 (HAS2) gene on the canine chromosome 13 to determine the number of duplications present in this region, wherein the number of duplications indicates the relative risk that the dog will develop Familial Shar-Pei fever.

2. The method of claim 1, wherein a copy number of >10 duplications indicates a high risk for Shar-Pei fever, a copy number of <5 indicates a low risk, and a copy number between 5-10 indicates an intermediary risk.

3. An isolated nucleic acid probe comprising a sequence present in a duplication on the canine chromosome 13 corresponding to the nucleic acid sequence SEQ ID NO:1, and wherein the nucleic acid probe hybridizes to a part of the nucleic acid sequence SEQ ID NO:1, or a sequence complementary thereto, under stringent conditions.

4. An isolated nucleic acid primer pair comprising a first primer and a second primer, wherein each of the first and second primers comprises a sequence present in a duplication on the canine chromosome 13 corresponding to the nucleic acid sequence SEQ ID NO:1, and wherein the primer pair amplifies a nucleic acid containing at least a part of said duplication.
